(19) United States
(12) Patent Application Publication
(10) Pub. No.: US 2018/0214550 A1
(43) Pub. Date: Aug. 2, 2018

(54) METHODS AND PHARMACEUTICAL COMPOSITIONS FOR ENHANCING NK CELL KILLING ACTIVITIES

(71) Applicants: INSERM (INSTITUT NATIONAL DE LA SANTÉ ET DE LA RECHERCHE MÉDICALE), Paris (FR); UNIVERSITÉ PARIS DIDEROT - PARIS 7, Paris (FR); ASSISTANCE PUBLIQUE-HÔPITAUX DE PARIS (APHP), Paris (FR); INSTITUT JEAN GODINOT, Reims (FR)

(72) Inventors: Armand BENSUSSAN, Paris (FR); Martine BAGOT, Paris (FR); Jérôme GIUSTINIANI, Reims (FR); Yacine MERROUCHE, Reims (FR); Adèle DE MASSON, Paris (FR); Anne MARIE-CARDINE, Paris (FR)

(21) Appl. No.: 15/741,944

(22) PCT Filed: Jul. 7, 2016
(86) PCT No.: PCT/EP2016/066099

§ 371 Date: Jan. 4, 2018

(30) Foreign Application Priority Data
Jul. 7, 2015 (EP) 15306109.8

(51) Int. Cl. A61K 39/395; A61K 39/39; C07K 16/28

(52) U.S. Cl. CPC A61K 39/39566; A61K 2039/572; C07K 16/2896; A61K 39/39

(57) ABSTRACT

The present disclosure relates to methods and pharmaceutical compositions for enhancing NK cell killing activities. In particular, the disclosure relates to a method of enhancing NK cell killing activities in a subject in need thereof comprising administering to the subject a therapeutically effective amount of a compound capable of stimulating CD245 on NK cells.

### FIELD OF THE INVENTION

The present invention relates to methods and pharmaceutical compositions for enhancing NK cell killing activities.

### BACKGROUND OF THE INVENTION

Natural Killer (NK) cells were identified over 40 years ago as a subset of lymphocytes able to spontaneously kill tumor cells in the absence of pre-stimulation (1-4). Present in most mammalian and avian species, NK cells play a critical role in the anti-tumor and anti-infectious immunity (5,6) and in reproduction (7). In humans, NK lymphocytes are phenotypically characterized by the expression of CD56, an isoform of the neural cell adhesion molecule, and by the absence of CD3 (8). NK cell cytotoxicity is tightly controlled by a balance between signals from the engagement of activating and inhibitory receptors (6,9). Upon contact with a target cell, integrins on the NK cell surface bind to adhesion molecules on the target cell and stabilize the cell-to-cell interaction (10). Binding of the integrin Lymphocyte Function-associated Antigen 1 (LFA-1) to Intercellular Adhesion Molecule 1 (ICAM-1) on target cells initiate an early signaling cascade in NK cells through activation of the guanine nucleotide exchange factor (GEF) domain-containing Vav1 and of p21-activated kinases (PAK) (11). This leads to cytoskeleton reorganization and aggregation of activating NK receptors (NKR) at the NK-target cell interface (12), referred to as the NK immune synapse (NKIS) (13). Engagement of activating NKRs leads to the recruitment of immunoreceptor tyrosine activation motifs (ITAM)-bearing adapter proteins. The two tyrosines in the ITAM are phosphorylated by Src-kinase family members, and phosphorylated ITAM form a binding site for the Src-homology domain 2 (SH2) domains of tyrosine kinases (14), triggering a signalling cascade responsible for granule polarization, degranulation, and cytolysis of the target cell. Activating NKR include members of the family of natural cytotoxicity receptors (such as CD245 (15), NKp44 (16), and NKp30 (17)), of the NKG2 family of C-type lectin receptors (NKG2D) (18), of the killer cell Ig-like receptors (KIRs) (19,20), of the Ig-like signaling lymphocytic activation molecule (SLAM) family (2B4) (21), and others such as CD160 (22,23). 4-1BB (CD137) is a costimulatory receptor expressed on T, B and NK cells (24) whose expression is triggered by engagement of Fc receptors on the NK cell surface, as is the case during antibody-dependent cell cytotoxicity (25). Stimulation of CD137 increases cetuximab-, rituximab-, and trastuzumab-dependent NK cell cytotoxicity in different cancer models (26-28). NK cell activation is dominantly suppressed if the inhibitory NKR bind to major histocompatibility complex (MHC) class I molecules on target cells (29). In humans, these receptors mainly belong to C-type lectin receptors, as the NKG2A heterodimer (30), or to the KIR superfamily of receptors (19,20). Unlike the activating KIRs, the inhibitory KIRs carry a long cytoplasmic tail bearing immunoreceptor tyrosine inhibition motifs (ITIM) sequences (31). The latter provide a specific binding site for the tandem SH2 domains of Src homology region 2-containing protein tyrosine phosphatase-1 (SHP-1) or SHP-2. SHP recruitment at the NKIS is able to block many of the key steps in the signalling cascade leading to cytolysis (32). CD245 was previously described as a unique surface antigen on the surface of human peripheral blood lymphocytes, recognized by the monoclonal antibody DY12 (33) However CD245 molecular and functional characteristics remain largely unknown.

### SUMMARY OF THE INVENTION

The present invention relates to methods and pharmaceutical compositions for enhancing NK cell killing activities. In particular, the present invention is defined by the claims.

### DETAILED DESCRIPTION OF THE INVENTION

The inventors combined immunological and proteomic approaches to identify CD245 as the unconventional myosin 18A, a highly conserved motor enzyme that plays a critical role in cytoskeleton organization and Golgi budding. NK cells from the blood and lung of healthy humans constitutively expressed myosin 18A. Myosin 18A localized to plasma membrane and cytoplasm of NK cells, and its membrane expression was activation-induced. Recruitment of myosin 18A on NK cells strongly enhanced both their cytotoxicity against P815 target cells coated with anti-CD335 (NKp46) or anti-CD337 (NKp30) and lymphokine-activated killer activity towards Epstein-Barr Virus (EBV)-infected B cells. Stimulation of myosin 18A induced CD137 surface expression on NK cells. Blocking of CD137L abrogated myosin 18A-induced NK cell cytotoxicity towards CD137L-expressing B lymphoma cells. Myosin 18A was associated with a phosphatase activity and able to bind Src homology region 2-containing protein tyrosine phosphatase-2 (SHP-2), a signal transducer of several NK receptors, and the p21-activated kinase 2 PAK2 involved in actin organization and formation of the NK immune synapse. The newly described NK activating receptor myosin 18A appears as a promising target in cancer immunotherapy.

Accordingly, one object of the present invention relates to a method of enhancing NK cell killing activities in a subject in need thereof comprising administering to the subject a therapeutically effective amount of a compound capable of stimulating CD245 on NK cells.

As used herein, “NK cells” refers to a sub-population of lymphocytes that is involved in non-conventional immunity. NK cells can be identified by virtue of certain characteristics and biological properties, such as the expression of specific surface antigens including CD56 and/or CD16 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response (“NK cell killing activities”). Any subpopulation of NK cells will also be encompassed by the term NK cells. Within the context of this invention “active” NK cells designate biologically active NK cells, including NK cells having the capacity of lysing target cells or enhancing the immune function of other cells. For instance, an “active” NK cell can be able to kill cells that express a ligand for an activating NK receptor and/or fail to express MHC/HLA antigens recognized by a KIR on the NK cell.

As used herein, the term “CD245” denotes the unconventional myosin 18A, unconventional myosin-XVIIIa or Myo18A. Exemplary human nucleic acid sequences are referenced in under NCBI Reference Numbers NM_078471.3 and NM_203318.1. Exemplary human amino acid sequences are referenced in under NCBI Reference Numbers NP_510880.2 and NP_976063.1. A further exemplary amino acid sequence includes SEQ ID NO:1 (FIG. 1B).

As used herein, the expression “compound that is capable of stimulation CD245 on NK cells” refers to any compound that is capable of engaging CD245 (i.e. binding to) and enhancing NK cell killing activities. The ability of the compound of the present invention to enhance NK cell killing activity may be determined by any assay well known in the art. Typically said assay is an in vitro assay wherein NK cells are brought into contact with target cells (e.g. target cells that are recognized and/or lysed by NK cells). For example, the compound can be selected for the ability to increase specific lysis by NK cells by more than about 20%, preferably with at least about 30%, at least about 40%, at least about 50%, or more of the specific lysis obtained at the same effector: target cell ratio with NK cells or NK cell lines that are contacted by the compound of the present invention, Examples of protocols for classical cytotoxicity assays are described, for example, in Pessino et al, J. Exp. Med, 1998, 188 (5): 953-960; Sivori et al, Eur J Immunol, 1999. 29:1656-1666; Brando et al, (2005) J. Leukoc. Biol. 78:359-371; El-Sherbiny et al, (2007) Cancer Research 67(18):8444-9; and Nolte-'t Hoen et al, (2007) Blood 109:670-673). Typically, NK cell cytotoxicity is determined by any assay described in the EXAMPLE. NK cell cytotoxicity may be measured by a classical in vitro chromium release test of cytotoxicity. Effector cells are typically fresh PB-NK from healthy donors. The target cells are typically the murine mastocytoma P815 cells or EBV-infected B cell lines. Accordingly, the compound of the present invention is selected if it causes an increase in the reactivity or cytoxicity of NK cells toward target cells (infected cells, tumor cells, pro-inflammatory cells, etc.), increased activation, activation markers (e.g. CD107 expression) and/or IFNgamma production in NK cells, and/or increased the frequency in vivo of such activated, reactive, cytotoxic and/or activated NK cells.

In some embodiments, the compound of the present invention is an antibody having specificity to CD245.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments.

As used herein, the term “specificity” refers to the ability of an antibody to detectably bind an epitope presented on an antigen, such as a CD245, while having relatively little detectable reactivity with non-CD245 proteins or structures (such as other proteins presented on NK cells, or on other cell types). Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a CD245 polypeptide). The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. Framework Regions (FRs) refer to amino acid sequences interposed between CDRs.

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin.

The term “Fab′” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH::VL heterodimer which is usually expressed from a gene fusion including VH and VL encoding genes linked by a peptide-encoding linker. “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with the appropriate antigenic forms (i.e. polypeptides of the present invention). The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant polypeptide of the present invention may be provided by expression with recombinant cell lines. Recombinant forms of the polypeptides may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods. Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In some embodiments, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans. In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. The amino acid sequence and structure of a single domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementarity Determining Region for “CDR1”; as “Complementarity Determining Region 2” or “CDR2” and as “Complementarity Determining Region 3” or “CDR3”, respectively. Accordingly, the single domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3.

In some embodiments, the antibody of the present invention comprises human heavy chain constant regions sequences but will not deplete NK cells to which they are bound and preferably do not comprise an Fc portion that induces antibody dependent cellular cytotoxicity (ADCC). As used herein, the term “depleting”, with respect to CD245-expressing cells means a process, method, or compound that can kill, eliminate, lyse or induce such killing, elimination or lysis, so as to negatively affect the number of CD245 expressing cells present in a sample or in a subject. The terms “Fc domain,” “Fc portion,” and “Fc region” refer to a C-terminal fragment of an antibody heavy chain, e.g., from about amino acid (aa) 230 to about aa 450 of human gamma heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, Md.). In some embodiments the antibody of the present invention does not lead, directly or indirectly, to the depletion of NK cells expressing CD245 polypeptides (e.g. do not lead to a 10%, 20%, 50%, 60% or greater elimination or decrease in number of CD245+NK cells). In some embodiments, the antibody of the present invention does not comprise an Fc domain capable of substantially binding to a FcγRIIIA (CD16) polypeptide. In some embodiments, the antibody of the present invention lacks an Fc domain (e.g. lacks a CH2 and/or CH3 domain) or comprises an Fc domain of IgG2 or IgG4 isotype. In some embodiments, the antibody of the present invention consists of or comprises a Fab, Fab′, Fab′-SH, F (ab′) 2, Fv, a diabody, single-chain antibody fragment, or a multispecific antibody comprising multiple different antibody fragments. In some embodiments, the antibody of the present invention is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. No. 6,194,551 by Idusogie et al.

In some embodiments, the hinge region of CHI is modified such that the number of cysteine residues in the hinge region is altered, e.g., increased or decreased. This approach is described further in U.S. Pat. No. 5,677,425 by Bodmer et al. The number of cysteine residues in the hinge region of CHI is altered to, for example, facilitate assembly of the light and heavy chains or to increase or decrease the stability of the antibody.

In some embodiments, the antibody of the present invention is modified to increase its biological half-life. Various approaches are possible. For example, one or more of the following mutations can be introduced: T252L, T254S, T256F, as described in U.S. Pat. No. 6,277,375 by Ward. Alternatively, to increase the biological half-life, the antibody can be altered within the CHI or CL region to contain a salvage receptor binding epitope taken from two loops of a CH2 domain of an Fc region of an IgG, as described in U.S. Pat. Nos. 5,869,046 and 6,121,022 by Presta et al.

Another modification of the antibodies herein that is contemplated by the present invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-poly ethylene glycol or polyethylene glycol-maleimide. In some embodiments, the antibody to be pegylated is an aglycosylated antibody. Methods for pegylating proteins are known in the art and can be applied to the human monoclonal antibodies of the present invention. See for example, EP 0 154 316 by Nishimura et al. and EP 0 401 384 by Ishikawa et al.

In some embodiments, the present invention provides a multispecific antibody comprising a first antigen binding site from an antibody of the present invention herein above and at least one second antigen binding site. Accordingly, the first antigen-binding site is used for recruiting a killing mechanism, i.e. increasing NK cytoxicity. Typically, the second antigen-binding site binds to an antigen that is expressed by a cancer cell or a cell infected by a virus, bacteria . . . . In some embodiments, the second antigen-binding site binds to an antigen on a human B cell, such as, e.g., CD19, CD20, CD21, CD22, CD23, CD80, CD138 and HLA-DR. In some embodiments, the second antigen-binding site binds to an antigen which is a tumor-associated antigen (TAA). Exemplary TAAs include carcinoembryonic antigen (CEA), prostate specific antigen (PSA), RAGE (renal antigen), α-fetoprotein, CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), ganglioside antigens, tyrosinase, gp75, c-Met, Marti, MelanA, MUM-1, MUM-2, MUM-3, HLA-B7, Ep-CAM or a cancer-associated integrin, such as α5β3 integrin. Exemplary formats for the multispecific antibody molecules of the present invention include, but are not limited to (i) two antibodies cross-linked by chemical heteroconjugation, one with a specificity to CD245 and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies. In some embodiments, the bispecific antibody is obtained or obtainable via a controlled Fab-arm exchange, typically using DuoBody technology. In vitro methods for producing bispecific antibodies by controlled Fab-arm exchange have been described in WO2008119353 and WO 2011131746 (both by Genmab A/S). In one exemplary method, described in WO 2008119353, a bispecific antibody is formed by “Fab-arm” or “half-molecule” exchange (swapping of a heavy chain and attached light chain) between two monospecific antibodies, both comprising IgG4-like CH3 regions, upon incubation under reducing conditions. The resulting product is a bispecific antibody having two Fab arms which may comprise different sequences. In another exemplary method, described in WO 2011131746, bispecific antibodies of the present invention are prepared by a method comprising the following steps, wherein at least one of the first and second antibodies is a antibody of the present invention: a) providing a first antibody comprising an Fc region of an immunoglobulin, said Fc region comprising a first CH3 region; b) providing a second antibody comprising an Fc region of an immunoglobulin, said Fc region comprising a second CH3 region; wherein the sequences of said first and second CH3 regions are different and are such that the heterodimeric interaction between said first and second CH3 regions is stronger than each of the homodimeric interactions of said first and second CH3 regions; c) incubating said first antibody together with said second antibody under reducing conditions; and d) obtaining said bispecific antibody, wherein the first antibody is an antibody of the present invention and the second antibody has a different binding specificity, or vice versa. The reducing conditions may, for example, be provided by adding a reducing agent, e.g. selected from 2-mercaptoethylamine, dithiothreitol and tris(2-carboxyethyl)phosphine. Step d) may further comprise restoring the conditions to become non-reducing or less reducing, for example by removal of a reducing agent, e.g. by desalting. Preferably, the sequences of the first and second CH3 regions are different, comprising only a few, fairly conservative, asymmetrical mutations, such that the heterodimeric interaction between said first and second CH3 regions is stronger than each of the homodimeric interactions of said first and second CH3 regions. More details on these interactions and how they can be achieved are provided in WO 2011131746, which is hereby incorporated by reference in its entirety.

Another modification of the antibodies that is contemplated by the present invention is a conjugate or a protein fusion of at least the antigen-binding region of the antibody of the present invention to serum protein, such as human serum albumin or a fragment thereof to increase half-life of the resulting molecule. Such approach is for example described in Ballance et al. EP0322094.

The antibody of the present invention may be produced by any technique known in the art, such as, without limitation, any chemical, biological, genetic or enzymatic technique, either alone or in combination. For example, knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies of the present invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

In some embodiments, the subject suffers from a cancer or an infectious disease. Accordingly, a further object of the present invention relates to a method of treating a cancer or an infectious disease in a subject in need thereof comprising administering to the subject a therapeutically effective amount of a compound capable of stimulating CD245 on NK cells.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. The methods of the present invention contemplate any one or more of these aspects of treatment.

As used herein, the term “cancer” has its general meaning in the art and includes, but is not limited to, solid tumors and blood borne tumors The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

As used herein the term “infectious disease” includes any infection caused by viruses, bacteria, protozoa, molds or fungi. In some embodiments, the viral infection comprises infection by one or more viruses selected from the group consisting of Arenaviridae, Astroviridae, Birnaviridae, Bromoviridae, Bunyaviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, Tymoviridae, Hepadnaviridae, Herpesviridae, Paramyxoviridae or Papillomaviridae viruses. Relevant taxonomic families of RNA viruses include, without limitation, Astroviridae, Birnaviridae, Bromoviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, and Tymoviridae viruses. In some embodiments, the viral infection comprises infection by one or more viruses selected from the group consisting of adenovirus, rhinovirus, hepatitis, immunodeficiency virus, polio, measles, Ebola, Coxsackie, Rhino, West Nile, small pox, encephalitis, yellow fever, Dengue fever, influenza (including human, avian, and swine), lassa, lymphocytic choriomeningitis, junin, machuppo, guanarito, hantavirus, Rift Valley Fever, La Crosse, California encephalitis, Crimean-Congo, Marburg, Japanese Encephalitis, Kyasanur Forest, Venezuelan equine encephalitis, Eastern equine encephalitis, Western equine encephalitis, severe acute respiratory syndrome (SARS), parainfluenza, respiratory syncytial, Punta Toro, Tacaribe, pachindae viruses, adenovirus, Dengue fever, influenza A and influenza B (including human, avian, and swine), junin, measles, parainfluenza, Pichinde, punta toro, respiratory syncytial, rhinovirus, Rift Valley Fever, severe acute respiratory syndrome (SARS), Tacaribe, Venezuelan equine encephalitis, West Nile and yellow fever viruses, tick-borne encephalitis virus, Japanese encephalitis virus, St. Louis encephalitis virus, Murray Valley virus, Powassan virus, Rocio virus, louping-ill virus, Banzi virus, Ilheus virus, Kokobera virus, Kunjin virus, Alfuy virus, bovine diarrhea virus, and Kyasanur forest disease. Bacterial infections that can be treated according to this invention include, but are not limited to, infections caused by the following: Staphylococcus; Streptococcus, including S. pyogenes; Enterococcl; Bacillus, including Bacillus anthracis, and Lactobacillus; Listeria; Corynebacterium diphtheriae; Gardnerella including G. vaginalis; Nocardia; Streptomyces; Thermoactinomyces vulgaris; Treponerna; Camplyobacter, Pseudomonas including aeruginosa; Legionella; Neisseria including N. gonorrhoeae and N. meningitides; Flavobacterium including F. meningosepticum and F. odoraturn; Brucella; Bordetella including B. pertussis and B. bronchiseptica; Escherichia including E. coli, Klebsiella; Enterobacter, Serratia including S. marcescens and S. liquefaciens; Edwardsiella; Proteus including P. mirabilis and P. vulgaris; Streptobacillus; Rickettsiaceae including R. fickettsfi, Chlamydia including C. psittaci and C. trachornatis; Mycobacterium including M. tuberculosis, M. intracellulare, M. folluiturn, M. laprae, M. avium, M. bovis, M. africanum, M. kansasii, M. intracellulare, and M. lepraernurium; and Nocardia. Protozoa infections that may be treated according to this invention include, but are not limited to, infections caused by leishmania, kokzidioa, and trypanosoma. A complete list of infectious diseases can be found on the website of the National Center for Infectious Disease (NCID) at the Center for Disease Control (CDC) (World Wide Web (www) at cdc.gov/ncidod/diseases/), which list is incorporated herein by reference. All of said diseases are candidates for treatment using the compositions according to the invention.

As used herein, the term “therapeutically effective amount” refers to an amount effective, at dosages and for periods of time necessary, to achieve a desired therapeutic result. A therapeutically effective amount of the compound of the present invention may vary according to factors such as the disease state, age, sex, and weight of the individual, and the ability of the compound of the present invention to elicit a desired response in the individual. A therapeutically effective amount is also one in which any toxic or detrimental effects of the antibody or antibody portion are outweighed by the therapeutically beneficial effects. The efficient dosages and dosage regimens for the compound of the present invention depend on the disease or condition to be treated and may be determined by the persons skilled in the art. A physician having ordinary skill in the art may readily determine and prescribe the effective amount of the pharmaceutical composition required. For example, the physician could start doses of the compound of the present invention employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. Alternatively, this property of a composition may be evaluated by examining the ability of the compound to induce cytotoxicity by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of a compound of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a compound of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled compound of the present invention, fragment or mini-antibody derived from the compound of the present invention. If desired, an effective daily dose of a pharmaceutical composition may be administered as two, three, four, five, six or more sub-doses administered separately at appropriate intervals throughout the day, optionally, in unit dosage forms. In some embodiments, the human monoclonal antibodies of the present invention are administered by slow continuous infusion over a long period, such as more than 24 hours, in order to minimize any unwanted side effects. An effective dose of a compound of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of a compound of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

The present invention also provides for therapeutic applications where a compound of the present invention is used in combination with at least one further therapeutic agent, e.g. for treating cancer. Such administration may be simultaneous, separate or sequential. For simultaneous administration the agents may be administered as one composition or as separate compositions, as appropriate. The further therapeutic agent is typically relevant for the disorder to be treated. Exemplary therapeutic agents include other anti-cancer antibodies, cytotoxic agents, chemotherapeutic agents, anti-angiogenic agents, anti-cancer immunogens, cell cycle control/apoptosis regulating agents, hormonal regulating agents, and other agents described below.

In some embodiments, the second agent is a natural ligand of an NK cell activating or an antibody that binds and activates an NK cell activating receptor other than CD245. In some embodiments, the agent is an agent that increases the presence of a natural ligand of an NK cell activating receptor on the surface of an target cell (e.g., infected cells, or tumor cells). NK cell activating receptors include, for example, NKG2D or activating KIR receptors (KIR2DS receptors, KIR2DS2, KIR2DS4). As used herein, the term “activating NK receptor” refers to any molecule on the surface of NK cells that, when stimulated, causes a measurable increase in any property or activity known in the art as associated with NK activity, such as cytokine (for example IFN-γ and TNF-α) production, increases in intracellular free calcium levels, the ability to target cells in a redirected killing assay as described, e.g. elsewhere in the present specification, or the ability to stimulate NK cell proliferation. The term “activating NK receptor” includes but is not limited to activating forms or KIR proteins (for example KIR2DS proteins), NKG2D, IL-2R, IL-12R, IL-15R, IL-18R and IL-21R. Examples of ligands that act as agonists at activating receptors include, e.g. IL-2, IL-15, IL-21 polypeptides. In some embodiments, the second antibody is specific for CD137. As used herein the term “CD137” has its general meaning in the art and may also be referred to as Ly63, ILA or 4-1BB. CD137 is a member of the tumor necrosis factor (TNF) receptor family. Members of this receptor family and their structurally related ligands are important regulators of a wide variety of physiologic processes and play an important role in the regulation of immune responses. CD137 is expressed by activated NK cells, T and B lymphocytes and monocytes/macrophages. The gene encodes a 255-amino acid protein with 3 cysteine-rich motifs in the extracellular domain (characteristic of this receptor family), a transmembrane region, and a short N-terminal cytoplasmic portion containing potential phosphorylation sites. Expression in primary cells is strictly activation dependent. The ligand for the receptor is TNFSF9. Human CD137 is reported to bind only to its ligand. Agonists include the native ligand (TNFSF9), aptamers (see McNamara et al. (2008) J. Clin. Invest. 1 18: 376-386), and antibodies.

In some embodiments, the compound of the present invention is used in combination with a chemotherapeutic agent. The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmo fur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; amino levulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and phannaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and phannaceutically acceptable salts, acids or derivatives of any of the above.

In some embodiments, the compound of the present invention is used in combination with a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names. In some embodiments, the targeted therapy consists of administering the subject with a tyrosine kinase inhibitor. The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo [3,4-f][1,6]naphthyridin-3 (2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In some embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

In some embodiments, the compound of the present invention is used in combination with an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ). Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behavior and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Combination compositions and combination administration methods of the present invention may also involve “whole cell” and “adoptive” immunotherapy methods. For instance, such methods may comprise infusion or re-infusion of immune system cells (for instance tumor-infiltrating lymphocytes (TILs), such as CC2+ and/or CD8+ T cells (for instance T cells expanded with tumor-specific antigens and/or genetic enhancements), antibody-expressing B cells or other antibody-producing or -presenting cells, dendritic cells (e.g., dendritic cells cultured with a DC-expanding agent such as GM-CSF and/or Flt3-L, and/or tumor-associated antigen-loaded dendritic cells), anti-tumor NK cells, so-called hybrid cells, or combinations thereof. Cell lysates may also be useful in such methods and compositions. Cellular “vaccines” in clinical trials that may be useful in such aspects include Canvaxin™, APC-8015 (Dendreon), HSPPC-96 (Antigenics), and Melacine® cell lysates. Antigens shed from cancer cells, and mixtures thereof (see for instance Bystryn et al., Clinical Cancer Research Vol. 7, 1882-1887, July 2001), optionally admixed with adjuvants such as alum, may also be components in such methods and combination compositions.

In some embodiments, the compound of the present invention is used in combination with radiotherapy. Radiotherapy may comprise radiation or associated administration of radiopharmaceuticals to a patient. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the compound of the present invention is used in combination with an antibody that is specific for a costimulatory molecule. Examples of antibodies that are specific for a costimulatory molecule include but are not limited to anti-CTLA4 antibodies (e.g. Ipilimumab), anti-PD1 antibodies, anti-PDLL antibodies, anti-TIMP3 antibodies, anti-LAG3 antibodies, anti-B7H3 antibodies, anti-B7H4 antibodies or anti-B7H6 antibodies.

In some embodiments, the second agent is an agent that induces, via ADCC, the death a cell expressing an antigen to which the second agent binds. In some embodiments, the agent is an antibody (e.g. of IgG1 or IgG3 isotype) whose mode of action involves induction of ADCC toward a cell to which the antibody binds. NK cells have an important role in inducing ADCC and increased reactivity of NK cells can be directed to target cells through use of such a second agent. In some embodiments, the second agent is an antibody specific for a cell surface antigens, e.g., membrane antigens. In some embodiments, the second antibody is specific for a tumor antigen as described above (e.g., molecules specifically expressed by tumor cells), such as CD20, CD52, ErbB2 (or HER2/Neu), CD33, CD22, CD25, MUC-1, CEA, KDR, αVβ3, etc., particularly lymphoma antigens (e.g., CD20). Accordingly, the present invention also provides methods to enhance the anti-tumor effect of monoclonal antibodies directed against tumor antigen(s). In the methods of the invention, ADCC function is specifically augmented, which in turn enhances target cell killing, by sequential administration of an antibody directed against one or more tumor antigens, and a compound of the present invention.

Accordingly, a further object relates to a method of enhancing NK cell antibody-dependent cellular cytotoxicity (ADCC) of an antibody in a subject in need thereof comprising administering to the subject the antibody, and administering to the subject a compound capable of stimulating CD245 on NK cells.

A further object of the present invention relates to a method of treating cancer in a subject in need thereof comprising administering to the subject a first antibody selective for a cancer cell antigen, and administering to the subject a compound capable of stimulating CD245 on NK cells.

A number of antibodies are currently in clinical use for the treatment of cancer, and others are in varying stages of clinical development. Antibodies of interest for the methods of the invention act through ADCC, and are typically selective for tumor cells, although one of skill in the art will recognize that some clinically useful antibodies do act on non-tumor cells, e.g. CD20. There are a number of antigens and corresponding monoclonal antibodies for the treatment of B cell malignancies. One popular target antigen is CD20, which is found on B cell malignancies. Rituximab is a chimeric unconjugated monoclonal antibody directed at the CD20 antigen. CD20 has an important functional role in B cell activation, proliferation, and differentiation. The CD52 antigen is targeted by the monoclonal antibody alemtuzumab, which is indicated for treatment of chronic lymphocytic leukemia. CD22 is targeted by a number of antibodies, and has recently demonstrated efficacy combined with toxin in chemotherapy-resistant hairy cell leukemia. Monoclonal antibodies targeting CD20, also include tositumomab and ibritumomab. Monoclonal antibodies useful in the methods of the invention, which have been used in solid tumors, include without limitation edrecolomab and trastuzumab (herceptin). Edrecolomab targets the 17-1 A antigen seen in colon and rectal cancer, and has been approved for use in Europe for these indications. Its antitumor effects are mediated through ADCC, CDC, and the induction of an anti-idiotypic network. Trastuzumab targets the HER-2/neu antigen. This antigen is seen on 25% to 35% of breast cancers. Trastuzumab is thought to work in a variety of ways: downregulation of HER-2 receptor expression, inhibition of proliferation of human tumor cells that overexpress HER-2 protein, enhancing immune recruitment and ADCC against tumor cells that overexpress HER-2 protein, and downregulation of angiogenesis factors. Alemtuzumab (Campath) is used in the treatment of chronic lymphocytic leukemia; colon cancer and lung cancer; Gemtuzumab (Mylotarg) finds use in the treatment of acute myelogenous leukemia; Ibritumomab (Zevalin) finds use in the treatment of non-Hodgkin's lymphoma; Panitumumab (Vectibix) finds use in the treatment of colon cancer. Cetuximab (Erbitux) is also of interest for use in the methods of the invention. The antibody binds to the EGF receptor (EGFR), and has been used in the treatment of solid tumors including colon cancer and squamous cell carcinoma of the head and neck (SCCHN).

Typically, the compound of the present invention is administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of an anti-CD245 antibody of the invention.

The invention will be further illustrated by the following figures and examples. However, these examples and figures should not be interpreted in any way as limiting the scope of the present invention.

### FIGURES

FIG. 1. Human NK cells express the long (α) and short (β) isoforms of myosin 18A

A. DY12 recognizes a unique 220-240 kDa protein at the cell surface of YT2C2 NK cells. Biotinylated YT2C2 leukemic cell lines were immunoprecipitated with DY12 mAb or control IgG1 (D6212) antibody. After revelation with horseradish peroxidase (HRP)-conjugated streptavidin, the immunoprecipitate was found to be a unique 220-240 kDa cell surface protein.

B. The sequence of the protein recognized by DY12 at the cell surface of YT2C2 NK cells corresponds to that of myosin 18A. Aminoacid sequence of the immunoprecipitate as determined by mass spectrometry (MS) analysis. Underlined are the sequences common to that of myosin 18A. In the list of 239 mass of tryptic peptides, 59 corresponded to those of myosin 18A, with a difference lower than 36 ppm from corresponding theoretical mass.

C. The protein recognized by DY12 is a target of anti-myosin 18A antibodies. YT2C2 cell lysates were immunoprecipitated using DY12 mAb or IgG1 control isotype and the immunoprecipitate was subjected to immunoblotting using polyclonal anti-myosin 18A antibodies. DY12 was shown to recognize the 230 kDa (myosin 18Aa, L-Myo18A) and 190 kDa (myosin 18Aβ, S-Myo18A) iso forms of myosin 18A.

D. DY12 recognizes the short isoform of myosin 18A at the cell surface of human peripheral blood lymphocytes. Fresh peripheral blood mononuclear cells (PBMCs) from healthy subjects were lysed, immunoprecipitated with DY12 or control IgG1 antibody, and immunoblotted using the anti-myosin18A polyclonal antibodies followed by HRP-conjugated goat anti-mouse antibodies. Whole PBMCs lysates were used as positive controls. The protein immunoprecipitated by DY12 in PBMCs from healthy subjects was the short isoform of myosin 18A (S-Myo18A).

E. DY12 recognizes the short and long isoforms of myosin 18A on fresh human lung lysates

Fresh, healthy, lung tissue from a human subject were lysed, immunoprecipitated with DY12 or control IgG1 antibody, and immunoblotted using the anti-myosin18A polyclonal antibodies followed by HRP-conjugated goat anti-mouse antibodies. Whole PBMCs lysates were used as positive controls. The proteins immunoprecipitated by DY12 were the short and long isoforms of myosin 18A (S-Myo18A).

FIG. 2. The recruitment of myosin 18A increases NK cell cytotoxicity

A, B, C, D, E, F. Myosin 18A-induced reverse cytotoxicity towards P815 mastocytoma cell lines. Cytotoxicity assays were performed according to a standard ⁵¹Cr-release method. Effector cells were freshly isolated (A, C, E) or IL-2 activated (B, D, F) PB-NK from healthy donors. The target cells were the murine mastocytoma P815 cells. P815 were preincubated with DY12 or control mAb, and anti-CD335 (NKp46) or anti-CD337 (NKp30) at 10 μg/ml. Assays at various Effector:Target (E:T) cell ratios with 10³ target cells were performed in triplicate.

G. The recruitment of myosin 18A increases the lymphokine-activated killer activity of human NK cells. Cytotoxicity assays were performed according to a standard ⁵¹Cr-release method. Effector cells were freshly IL-2 activated PB-NK from healthy donors. The target cells were the Epstein-Barr Virus (EBV)-infected B cell lines. Target cells were preincubated with DY12 control F(ab′)2 or control F(ab′)2 Ab at 10 μg/ml. Assays at various Effector:Target (E:T) cell ratios with 10³ target cells were performed in triplicate.

H. The recruitment of myosin 18A increases NK cell degranulation in the presence of target tumor cells.

Effector cells were freshly isolated PB-NK from healthy donors incubated for 1 h with DY12 or control IgG1 antibody at a concentration of 10 μg/ml, followed by crosslinking with rabbit anti-mouse IgG 10 μg/ml. The target cells were then added in a final volume of 100 μl/well at various effector/target ratios. After 4 h of culture at 37° C. in presence of PE-Cy7 anti-CD107a antibody, cells were washed and CD107a expression was measured on live CD3⁻ CD56⁺ NK cells by flow cytometry.

FIG. 3. Myosin 18A-induced NK cell cytotoxicity is 4-1BB (CD137)-dependent

Blocking the CD137-CD137L interaction with human CD137L polyclonal antibodies completely abrogates the CD245-induced NK cell degranulation in the presence of RAJI cells. Effector cells were freshly isolated PB-NK from healthy donors incubated for 1 h with DY12 or control IgG1 antibody at a concentration of 10 μg/ml, followed by crosslinking with rabbit anti-mouse IgG 10 μg/ml. The target cells were then added in a final volume of 100 μl/well at various effector/target ratios, in the presence or not of human CD137L polyclonal antibodies 10 μg/ml. After 4 h of culture at 37° C. in presence of PE-Cy7 anti-CD107a antibody, cells were washed and CD107a expression was measured on live CD3⁻CD56⁺NK cells by flow cytometry.

### EXAMPLE

Methods

Cells

Peripheral blood mononuclear cells (PBMC) were isolated from heparinized venous blood obtained from healthy donors by density gradient centrifugation over lymphocytes separation medium (PAA Laboratories/GE Healthcare Europe, Vélizy-Villacoublay, France). Fresh NK cells from the peripheral blood (PB-NK) cells were isolated by magnetic-activated cell sorting (MACS) using the NK cell isolation kit according to the manufacturers' recommendations (Miltenyi Biotec, Bergisch Gladbach, Germany). PB-NK cell purity was shown to be >90% as assessed by flow cytometry. YT2C2 NK cell lines (purchased from ATCC, Manassas, USA), Epstein-Barr Virus (EBV)-infected B cell lines (locally produced (34)) and RAJI cells (a Burkitt-lymphoma B-cell line, ATCC) were cultured in Roswell Park Memorial Institute (RPMI) 1640 medium supplemented with penicillin/streptomycin, L-glutamine, and 10% heat-inactivated fetal calf serum (FCS) (Perbio Science, Villebon-sur-Yvette, France).

Flow Cytometry Analysis of CD245 Expression by PBMCs

The monoclonal antibodies (mAbs) to human antigens used in flow cytometry assays in this study were the following: anti-CD3, anti-CD4, anti-CD8, anti-CD20, anti-CD56, anti-CD279 (Programmed cell Death (PD)-1), anti-CD197 (C-C chemokine receptor type 7 (CCR7)), anti-γδ T-cell receptor mAb (Milteniy), and anti-CD245 mAb (DY12, mouse IgG1k, locally produced). Irrelevant isotype-matched Abs were used as negative controls. Fluorescein isothiocyanate (FITC), allophycocyanin (APC)- or R-phycoerythrin (RPE)-conjugated goat anti-mouse IgG or IgM (Beckman Coulter, Brea, USA) were used as secondary reagents. Cells were phenotyped by indirect immunofluorescence. Briefly, the cells were incubated with the specific mAb for 30 min at 4° C., washed twice in phosphate buffer saline (PBS) (Life Technologies, Carlsbad, USA), and further incubated with the appropriated FITC- or RPE-labeled secondary Abs. Cells were washed and analyzed by flow cytometry on a FC 500 analyzer (Beckman Coulter). In some experiments, whole PBMC were stimulated with recombinant human IL-2 100 IU/ml (Peprotech France, Neuilly-sur-Seine, France) 72 h before cell labeling for flow cytometry analysis.

Immunohistochemistry

Formalin-fixed and paraffin-embedded lung biopsies and PB-NK from the peripheral blood of healthy subjects were analyzed for CD245 expression using a standard peroxidase method. PB-NK were pre-incubated or not with recombinant human IL-15 10 ng/ml overnight. Mouse anti-human CD245 antibody (DY12, locally produced), or monoclonal mouse anti-human granzyme B (clone GrB-7, DAKO) was used as the primary antibody followed by peroxidase-conjugated anti-mouse antibody revealed with the avidin-streptavidin peroxidase (LSAB kit, Dako, Les Ulis, France). The peroxidase reaction was then developed using 3-amino-9-ethyl carbazole substrate for 5 to 8 minutes.

Cell Surface Biotinylation

Cells were biotinylated by a sulfosuccinimidobiotin (Sulfo-NHS-biotin, Pierce, Rockford, USA) procedure. Briefly, after three washes in PBS, cells were suspended at 10×10⁶/ml in PBS) and 1 mg/ml of Sulfo-NHS-biotin. After a 30-min incubation at 4° C., cells were washed three times with complete medium.

Immunoprecipitation and Western Blot

YT2C2 biotinylated cells were lysed and incubated with DY12 or D6212 control IgG1 Ab followed by protein G-Sepharose beads. The precipitated proteins were washed, separated by SDS-8% PAGE and blotted onto a nitrocellulose membrane (Millipore, Bedford, USA). The membrane was blocked for 1 h with 5% dried milk in PBS plus 0.05% Tween-20, and the protein bands were developed with horseradish peroxidase (HRP)-conjugated streptavidin and enhanced chemiluminescent (ECL) reagents (Amersham Biosciences, GE Healthcare Europe).

Immunoblotting

Immunoprecipitation using DY12 or D6212 control Ab was performed on YT2C2 cell lysates, freshly isolated human NK cells or fresh human lung, as described above. The immunoprecipitates were resolved by 8% sodium dodecylsulfate polyacrylamide gel electrophoresis (SDS-PAGE), blotted onto a nitrocellulose membrane and subjected to immunoblot analysis using rabbit polyclonal anti-myosin 18A (Protein Tech Group, Manchester, UK), anti-SHP2 or anti-PAK2 polyclonal Abs (Cell Signaling Technologies, Beverly, USA). HRP-conjugated goat anti-rabbit Abs (Jackson ImmunoResearch Laboratories, West Grove, USA) were used as secondary Abs, and the immunoreactive proteins were visualized using an ECL kit (Amersham Biosciences). Whole YT2C2 cell lysates were used as positive controls.

Mass Spectrometry (MS)

After immunoprecipitation with DY12 mAb, the band was cut with a scalpel from the nitrocellulose. The piece of blot was then processed for MS analysis without chemical treatment as previously described (35,36). The band was digested with trypsin and MS analysis was carried out using a MALDI TOF/TOF ABI 4800 (Applied Biosystems, Foster City, USA). The masses obtained by MS-MALDI were analyzed using the Expasy database and software [http://www.expasy.org] and a local Visual Basic for Applications (VBA) software (Microsoft Excel, Microsoft, Redmond, USA).

Cytotoxicity Assays

Cytotoxicity assays were performed according to a standard ⁵¹Cr-release method. Effector cells were fresh PB-NK from healthy donors. The target cells were the murine mastocytoma P815 cells or EBV-infected B cell lines. Target cells were labeled with 100 μCi of Na51CrO4 for 90 min at 37° C., and washed three times in RPMI 1640 medium containing 10% FCS. The target cells were then plated in 96-well V-bottom microtiter plates (Greiner BioOne, Courtaboeuf, France).

In redirected cytotoxicity assays against P815 cell lines, PB-NK cells were pre-activated or not by 72h culture in the presence of recombinant human IL-2 (100 international units (IU)/ml). P815 were then preincubated with DY12 or control mAb, and anti-CD335 (NKp46) or anti-CD337 (NKp30) at 10 μg/ml. Assays at various Effector:Target (E:T) cell ratios with 10³ target cells were performed in triplicate.

In lymphokine-activated killer assays against EBV-infected B cell lines, PB-NK were preactivated for 24h or not in the presence of recombinant human IL-15 10 ng/ml (Peprotech). The effector cells were then added in a final volume of 150 μl/well in the presence of DY12 mAb or control IgG1 antibody (10 μg/ml).

After 4 h of culture at 37° C., the plates were spun down and 100 μl of the cell supernatant were collected from each well. The ⁵¹Cr release was quantified using a gamma-counter (Packard Instrument Company, Meriden, USA). The percentage of specific lysis was calculated as follows: % Specific lysis=[(Sample cpm−Spontaneous Lysis Control cpm)/Maximum Lysis Control cpm−Spontaneous Lysis Control cpm)]×100. The lysis was considered significant if >10% of the maximum cell lysis.

Flow Cytometry Analysis of NK Cell Activating Receptors Expression

The expression of NK cell activating receptors was assessed on freshly purified NK cell lines after 1 h in vitro stimulation of Myo18A with DY12 or control IgG1 antibody at a concentration of 10 μg/ml, washing and crosslinking with rabbit anti-mouse IgG (Jackson ImmunoResearch Laboratories) 10 μg/ml. Cells were then washed and labeled with Fixable Viability Stain 450 (Becton Dickinson, Franklin Lakes, USA) and the following antibodies to human cell surface antigens: APC-conjugated anti-CD137, PE-conjugated anti-NKG2D, FITC-conjugated anti-DNAX Accessory Molecule-1 (DNAM-1, CD226), PE-conjugated anti-CD160 (Becton Dickinson), PE-conjugated anti-NKp30 (CD337), anti-NKp44 (CD336), and anti-NKp46 (CD335) (Beckman-Coulter). Cells were washed and analyzed on a Canto II Cytometer (Becton Dickinson).

CD137L Expression by Target B Cell Lines

RAJI and EBV-infected B cell lines (34) were cultured as described above, washed and stained with Fixable Viability Stain 450 (Becton Dickinson) and PE-conjugated anti-CD137L (Becton Dickinson) for flow cytometry analysis.

Cytotoxicity Against RAJI, EBV and Blocking of the CD137/CD137L Interaction

Freshly isolated PB-NK cells were pre-activated or not by 12h culture in the presence of recombinant human IL-15 10 ng/ml (Peprotech), washed, and incubated for 1 h with DY12 or control IgG1 antibody at a concentration of 10 μg/ml, washed again and crosslinked with rabbit anti-mouse IgG (Jackson ImmunoResearch Laboratories) 10 μg/ml. The target cells were then added in a final volume of 100 μl/well at various effector/target ratios. After 4 h of culture at 37° C. in presence of PE-Cy7 anti-CD107a (Becton Dickinson), cells were washed and prepared for flow cytometry analysis. In some experiments, human 4-1BB Ligand/TNFSF9 Affinity Purified Polyclonal Ab (RnD systems, Minneapolis, USA) was added to the culture at a final concentration of 10 μg/ml to block the CD137/CD137L interaction.

Analysis

Flow cytometry analysis were carried out using FlowJo software. All values are expressed as means. Values are plotted with their mean and standard deviation and compared between groups with Prism software (Graph Pad) by two-tailed Mann-Whitney U test to compare continuous variables in 2 sample groups. p≤0.05 was considered statistically significant.

Results

Human NK Cells Express the Long (α) and Short (β) Isoforms of Myosin 18A

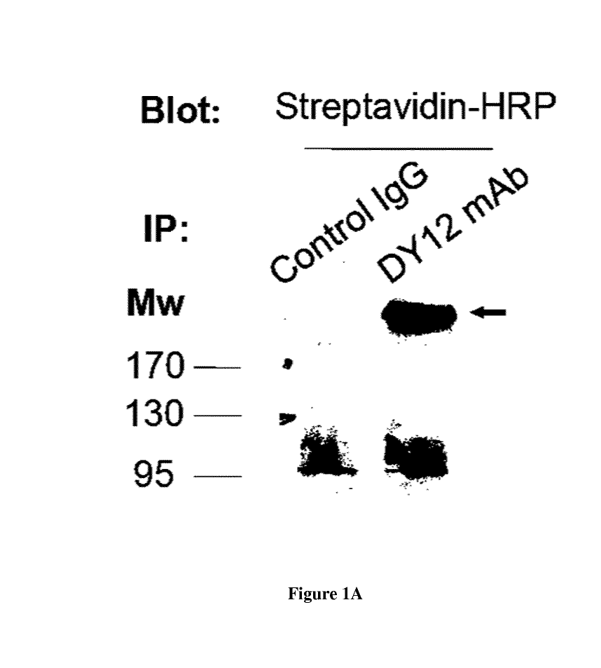

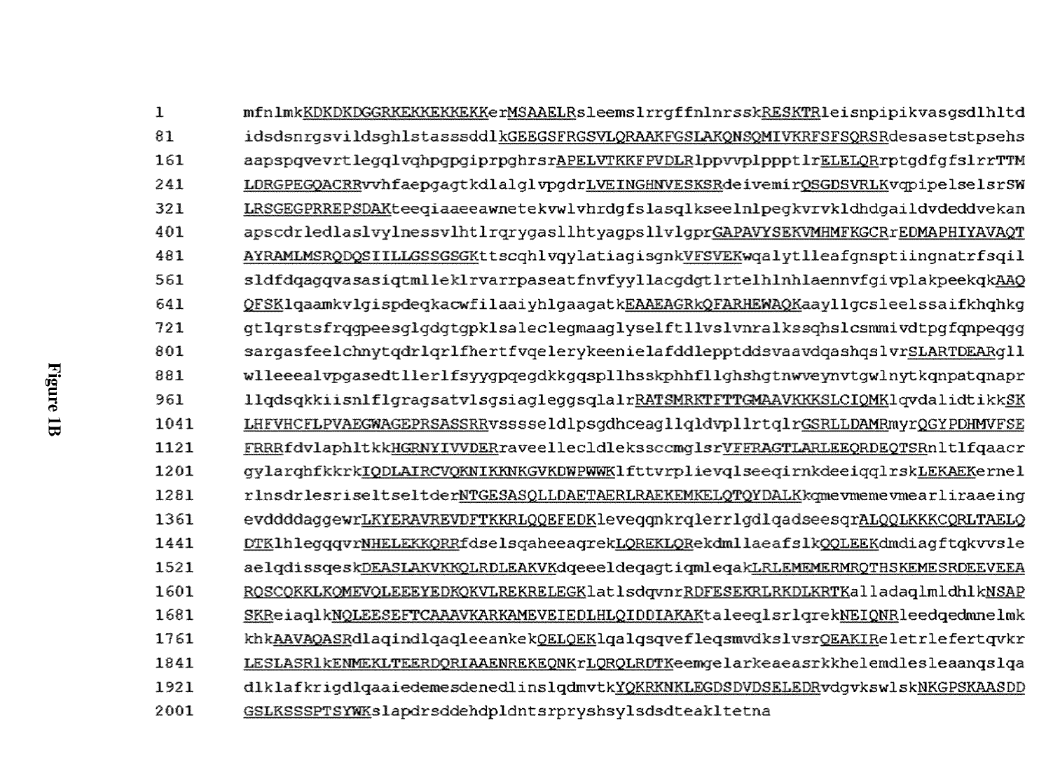

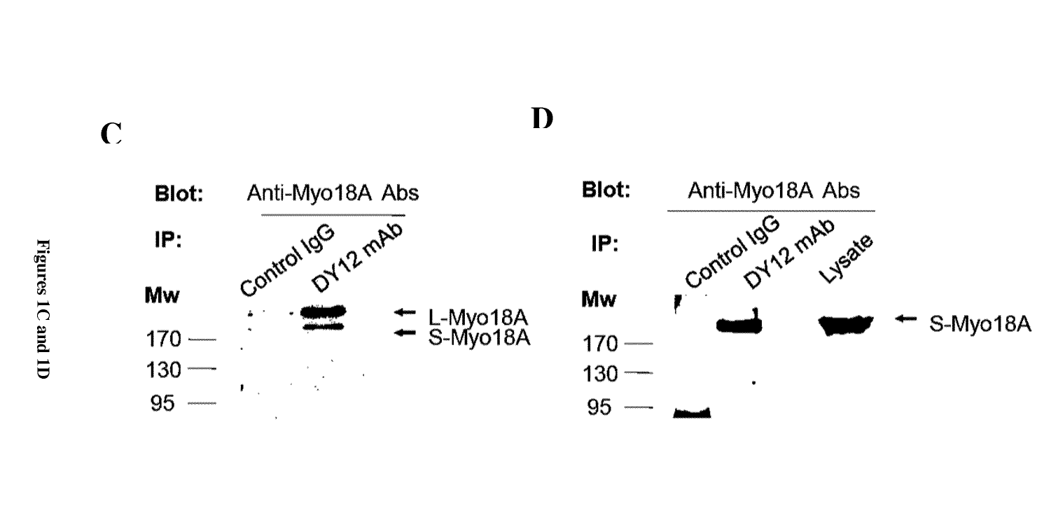

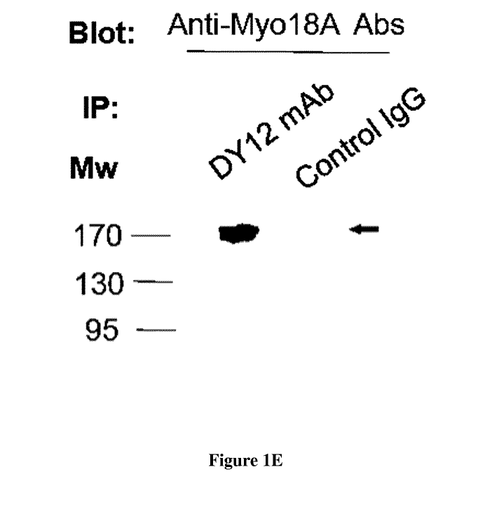

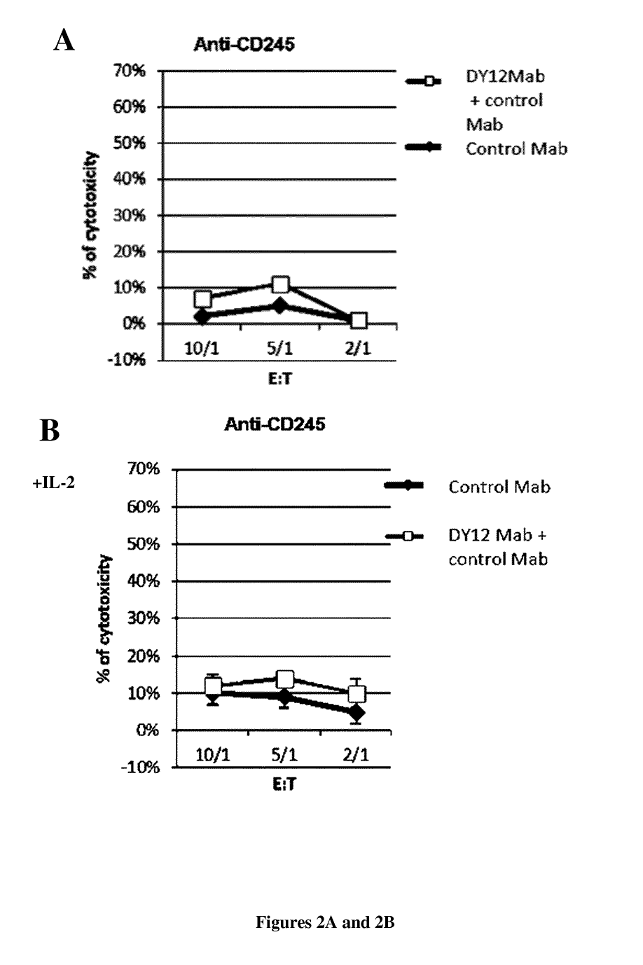

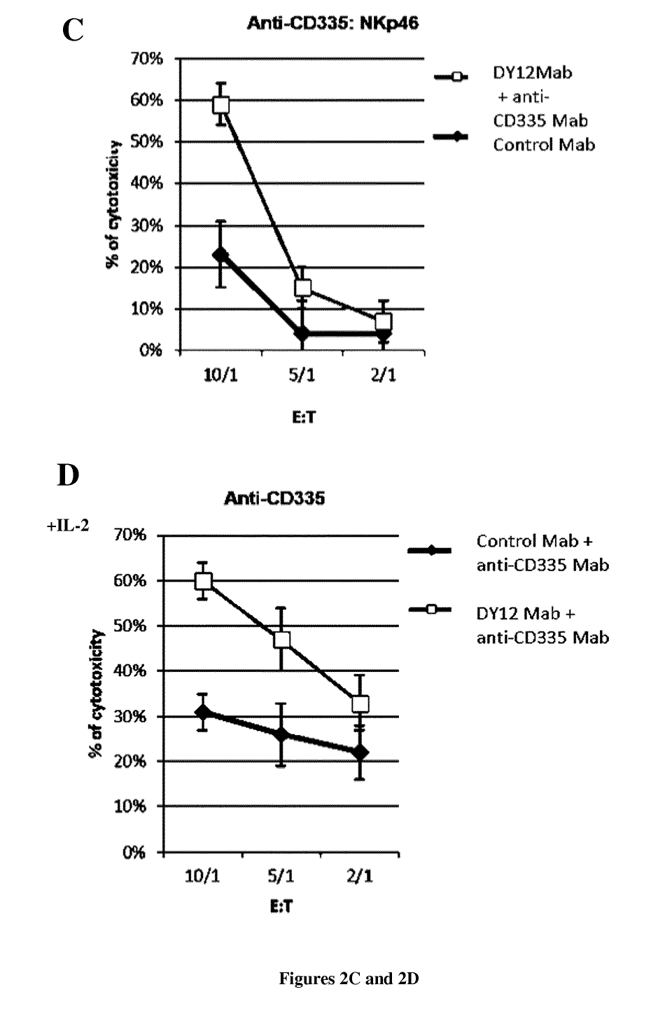

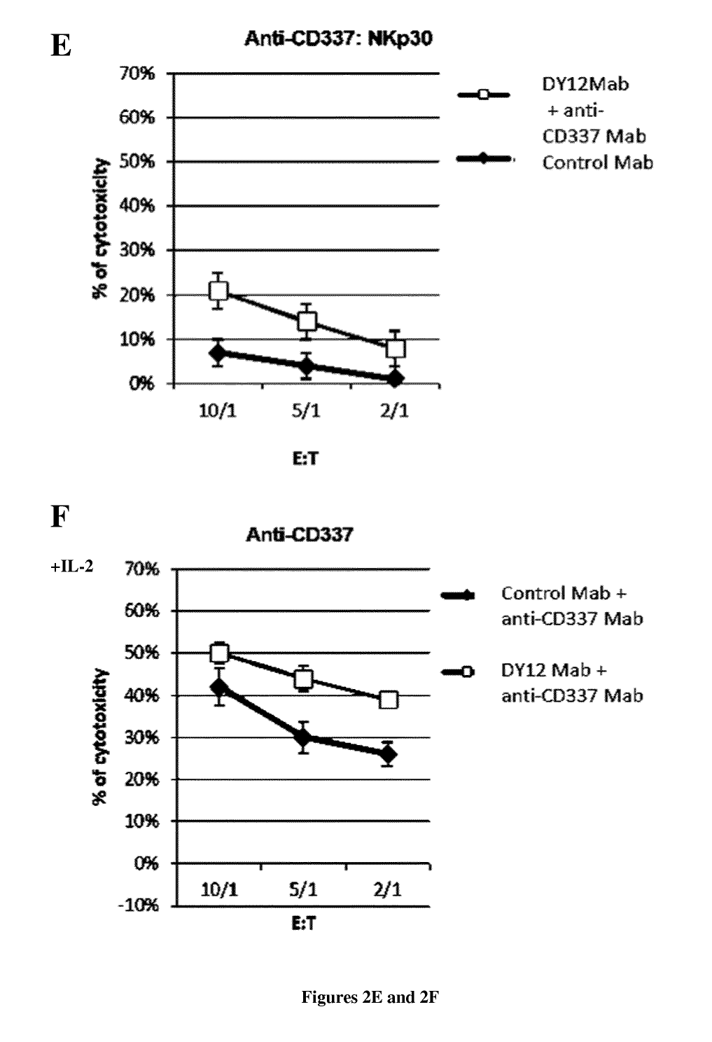

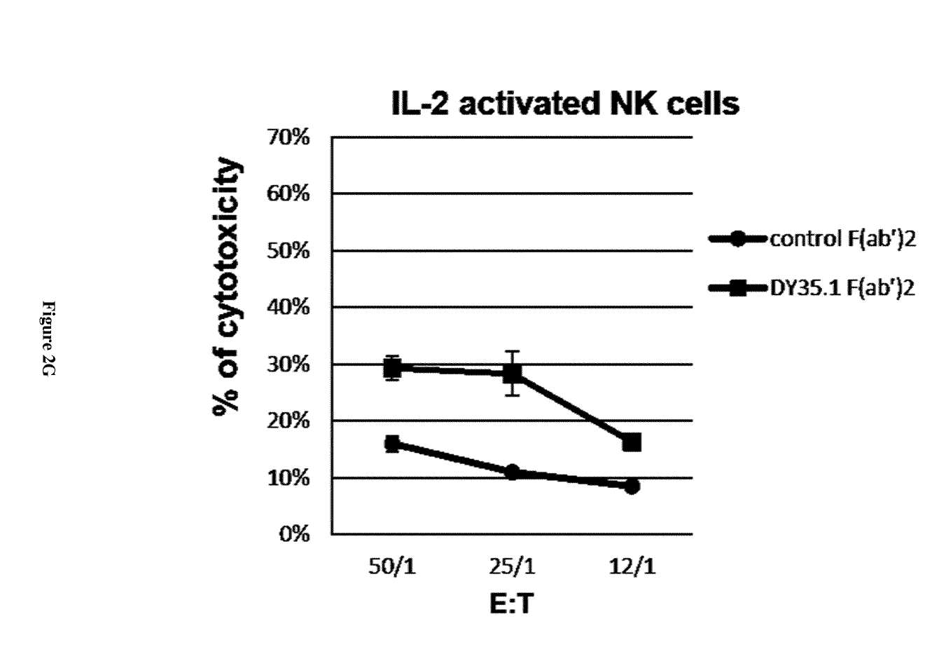

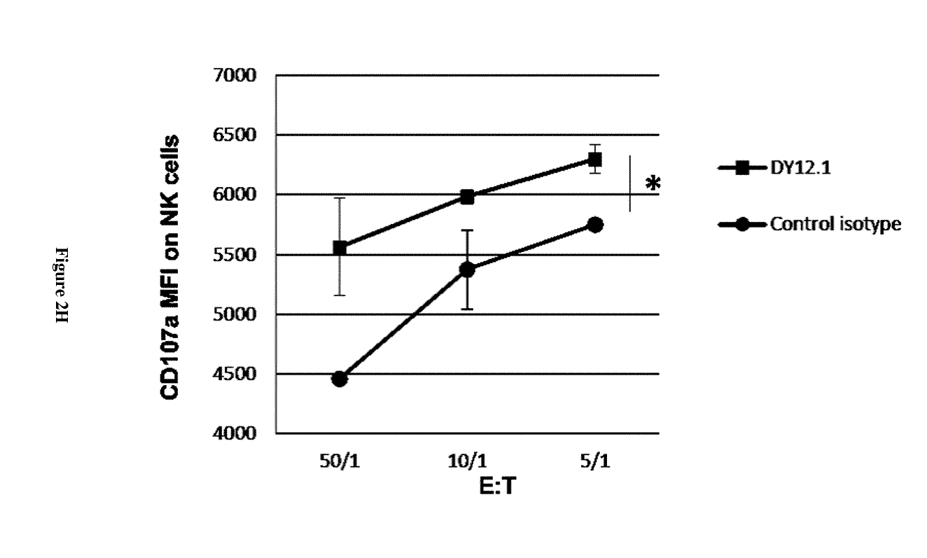

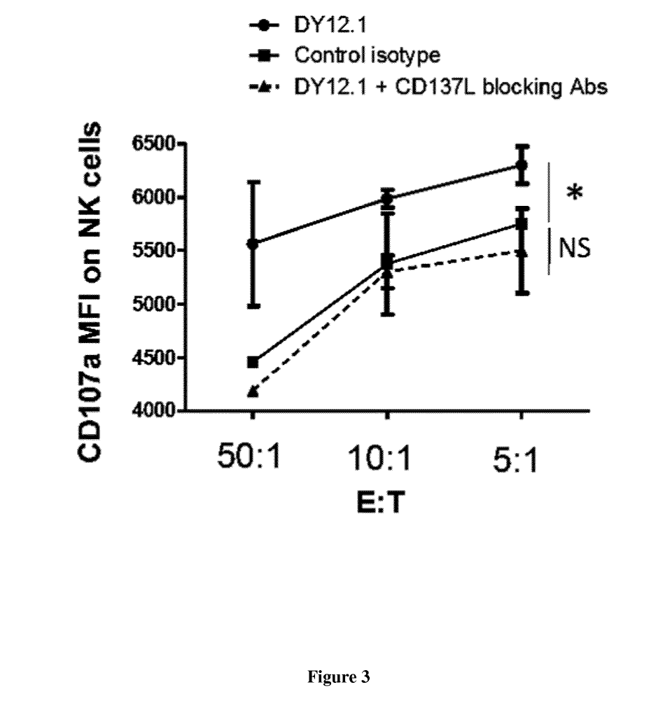

We previously described CD245 as a surface antigen expressed by human hematopoietic cells, recognized by the monoclonal antibody DY12 (33). To determine the molecular characteristics of CD245, we immunoprecipitated biotinylated YT2C2 leukemic cell lines with DY12 mAb or control IgG1 antibody. As shown in FIG. 1A, after revelation with HRP-conjugated streptavidin, the immunoprecipitate was found to be a unique 220-240 kDa cell surface protein. To further characterize this cell surface protein, the band was cut with a scalpel from the nitrocellulose, digested with trypsin and then processed for mass spectrometry (MS) analysis as previously described (36). In the list of 239 masses of tryptic peptides, 59 corresponded to those of myosin 18A, with a difference lower than 36 ppm from corresponding theoretical mass (FIG. 1B). To confirm that CD245 expressed at the cell surface of YT2C2 cell lines was the unconventional myosin 18A, YT2C2 cell lysates were immunoprecipitated using DY12 mAb or IgG1 control isotype and the immunoprecipitate was subjected to immunoblotting using polyclonal anti-myosin 18A antibodies. DY12 was shown to recognize the 230 kDa (myosin 18Aα) and 190 kDa (myosin 18Aβ) isoforms of myosin 18A (FIG. 1C). Thus, CD245 expressed at the cell surface of human YT2C2 NK cell line is the bona fide myosin 18A. In order to further investigate if CD245 expressed in vivo on human hematopoietic cells was myosin 18A, fresh PBMCs from healthy subjects were lysed, immunoprecipitated with DY12 or control IgG1 antibody, and immunoblotted using the anti-myosin18A polyclonal antibodies followed by HRP-conjugated goat anti-mouse antibodies. Whole PBMCs lysates were used as positive controls. The protein immunoprecipitated by DY12 in PBMCs from healthy subjects was the short isoform of myosin 18A (FIG. 1D). In conclusion, these results confirm that CD245 expressed on human hematopoietic cells is the unconventional myosin 18A. NK cells expressed the p190 and p230 isoforms. p230 was shown to be the main isoform expressed at the NK cell surface of YT2C2 cell lines. By contrast, p190, not p230, was found in whole human PBMC lysates. These data are consistent with previous studies in mice that showed that myosin 18Aα (p230) and β (p190) had different subcellular localizations, the former colocalizing with the endoplasmic reticulum and Golgi structures (37). We confirmed these results on fresh human lung lysates, showing that DY12 recognized the 2 isoforms of myosin 18A in the human lung (FIG. 1E).

Myosin 18A/CD245 Expression at the Cell Surface of Peripheral Blood Lymphocytes is Constitutive and Increased by Activation

To investigate the expression of Myo18A/CD245 on various subsets of peripheral blood lymphocytes in vivo, live PBMCs from healthy subjects were isolated and subjected to flow cytometry analysis using fluorochrome-conjugated DY12 mAb and anti-CD3, CD4, CD8, CD20, CD56, γδ TCR (T-cell receptor) mAbs. All peripheral blood lymphocyte subsets expressed Myo18A/CD245 at various degrees. Most CD3⁺ T cells, γδ T cells, CD56⁺ (i.e., NK cells, but also the numerically minor γδ T and NKT peripheral blood lymphocyte subsets), and half of the CD20⁺ B cells expressed Myo18A/CD245. CD245 expression was associated, although to a lesser extent, with that of CCR7, a chemokine receptor expressed in T, B cells and CD56^{bright} NK cells (38) involved in lymph node homing (39). CD56^{bright} cells expressed CD245 at higher levels than CD56^{dim} cells. After IL-2 activation of whole PBMCs, nearly all lymphocytes expressed CD245. CD245 mean fluorescence intensity increased between 3 and 8-fold after IL-2. By contrast, using a polyclonal antibody against surfactant protein A-receptor (SP-R)-210 that was previously shown to detect myosin 18A (40), Samten et al. found that myosin 18A was expressed by a very small fraction of peripheral blood CD3+ lymphocytes. The percentage of CD3⁺ cells expressing myosin 18A was increased five to 10 fold in 48 h M. tuberculosis-stimulated PBMCs (41).

Recruitment of Myosin 18A on Peripheral Blood NK Cells Increases NK Cell Reverse Cytotoxicity Towards Mastocytoma Cell Lines

To confirm the expression of CD245 by human NK cells from the peripheral blood, freshly isolated PB-NK from healthy donors were assessed for CD245 expression by immunohistochemistry using the DY12 mAb. Anti-granzyme B antibodies were used as positive control. PB-NK expressed myosin 18A at the cell membrane and in the cytoplasm in steady state.

SP-A, a ligand for Myo18A, has previously been shown to stimulate the anti-tumor immunity in vivo in a NK cell-dependent manner (42). We investigated whether the stimulation of Myo18A was able to modulate the NK cell cytotoxicity against tumor cells. As shown in FIG. 2A, B, C, D, E, F, NK cells stimulated with DY12 alone exhibited poor cytotoxic activity. By contrast, DY12 stimulation strongly enhanced NKp46- and NKp30-induced cell cytotoxicity against the P815 murine mastocytoma cell lines. On average, stimulation with DY12 increased NKp46- and NKp30-induced cell cytotoxicity by 69% and 283%, respectively in freshly isolated NK cells; and by 75% and 39% in IL-2 activated NK cells. These data identify CD245 as a strong activator of NK cell anti-tumor activity triggered by natural cytotoxicity receptors.

Myo18A Recruitment Increases the IL-2 Activated NK Cell Cytotoxicity Towards Epstein Barr Virus (EBV)-Infected B Cells

Because (i) NK cells play a critical and first-line role in the antiviral immune defense (5), (ii) human NK cells express high levels of myosin 18A, (iii) myosin 18A cell surface expression is induced by IL-2 in NK cells and (iv) myosin 18A has been shown to be a receptor for SP-A (40), that is involved in viral clearance in the human lung (43), we asked whether engagement of Myo18A on the NK cell surface was able to regulate their IL-2-activated killer activity against virally infected cells. Engagement of Myo on the surface of IL-2 activated PB-NK from healthy subjects increased their cytotoxic activity against EBV-infected B cell lines on average by 110% (83-158%) (FIG. 2G). The recruitment of Myo18A did not significantly increase the formation of conjugates between EBV-infected B cells and NK cells (data not shown), but increased PB-NK cell degranulation in the presence of RAJI cells by 25% in the 50/1 ratio (FIG. 2H). These data suggest that Myo18A plays a role in the lymphokine-activated killer cell activity and antiviral function of human NK cells.

NK Cell Cytotoxicity Induced by the Recruitment of Myosin 18A is 4-1BB-Dependent

To further understand the mechanism by which recruitment of CD245 increases NK cell cytotoxicity against tumor cell lines, we studied the expression of activating NK cell receptors after engagement of CD245 by DY12 mAb or control isotype. CD245 recruitment did not induce any significant change in the expression of NKp30, NKp44, NKp46 and NKG2D. Nor did it have significantly impact on the expression of DNAM1 or CD160 (data not shown). By contrast, CD245 stimulation increased the expression of CD137 (4.1BB) by on average 94% (56-156%). As shown above, CD245 stimulation increased the NK cell cytotoxicity against EBV-infected B cell lines (FIG. 2G) and RAJI B cells (FIG. 2H). B-cell lymphoma cells have previously been shown to express the CD137 ligand, CD137L (44). We confirmed that the target cells, EBV-infected B cells and RAJI cells, expressed CD137L. Blocking the CD137-CD137L interaction with human 4-1BB ligand polyclonal Ab completely abrogated the CD245-induced NK cell degranulation in the presence of RAJI cells (FIG. 3). By contrast, no significant increase in the NK cell degranulation was induced by DY12 in presence of Sezary cells that do not express CD137L (4-1BBL) (data not shown). Altogether, these data suggest that NK cell cytotoxicity induced by the recruitment of myosin 18A is 4-1BB-dependent.

Myosin 18A Interacts with PAK-2 and SHP-2, Two Key Signal Transducers of the NK Cell Activation and Degranulation

As shown previously, recruitment of Myo18A enhanced NK cell cytotoxicity against tumor cells and virally infected cell lines. NK cell cytotoxicity is dependent on cytoskeleton reorganization, to create the NK immune synapse first (13), and then to allow the polarization and exocytosis of the cytolytic granules (45,46). Myo18A has been shown to play a role in the cytoskeleton organization and to interact with PAK-2 in epithelial cell lines (47). To further elucidate the mechanisms by which CD245 stimulation increased NK cell degranulation and lysis of target cells, we immunoprecipitated YT2C2 NK cell lines with DY12 or control IgG1 isotype and subjected the immunoprecipitate to immunoblotting using anti-PAK2 antibody. Whole YT2C2 were used as positive controls.PAK2 immunoprecipitated with Myo18A in YT2C2 cells. These data show, for the first time, that Myo18A interacts with PAK2 in NK cells, and identify the PAK2 kinase as a potential signal transducer of the Myo18A-induced NK cell cytotoxicity.

As previously demonstrated (33), CD245 was shown to exhibit spontaneous phosphatase activity in the NK YT2C2 cell line. Among the main phosphatases involved in the signal transduction from NK receptors are the Src-homology domain-containing phosphatases (SHP) that, through their SH2 domains, interact with phosphorylated tyrosine residues on other proteins (32,48-50). In particular, SHP-2 regulates NK cell function (50). We thus investigated whether Myo18A was able to recruit SHP-2. Immunoprecipitation of YT2C2 cell lysates with DY12 antibody and further immunoblotting using anti-SHP-2 Ab revealed the association of Myo with the phosphatase SHP-2. Thus, SHP-2 may be involved in the signal transduction from the Myo18A activating receptor.

### Discussion

In the present work, we identified CD245, a human cell surface antigen expressed on peripheral blood lymphocytes, as the unconventional myosin 18A (Myo18A), a highly conserved motor enzyme involved in cytoskeleton organization and Golgi budding (51-54). We identified Myo18A/CD245 as a crucial human NK cell activating receptor, whose cell surface expression is induced by IL-2. Myo18A stimulation was able to increase NK cell degranulation and cytotoxicity towards virally infected and tumor B cells. We found that Myo18A stimulation was able to induce CD137 expression at the NK cell surface and that the Myo18A-induced NK cell cytotoxicity was dependent on the CD137/CD137L interaction. Last, Myo18A was able to interact with SHP-2, a phosphatase with a key role in the signal transduction of NK cell receptors (50) and PAK-2, a serine/threonine kinase that controls the cytoskeletal organization. These entirely novel molecular and functional data on a newly described NK cell activating receptor have broad potential applications.

The unconventional myosin 18A (Myo18A) is a member of the myosin superfamily of motor enzymes. Myosins generally contain a conserved catalytic head that catalyzes ATP hydrolysis and binds F-actin, thus promoting motility. The first myosin, M2, was discovered in muscle extracts and is referred to as conventional myosin, whereas other classes, including class 18, are called unconventional myosins (55). Myosin 2A is required for cytolytic granule exocytosis in human NK cells (56). Class 18 myosins have been involved in fundamental tissular processes in mammalians, including epithelial cell migration (47), stromal cell differentiation (57) and tumor suppression (58-60). In humans and mice, myosin 18A is expressed in hematopoietic cells as two splice variants, referred to as α (230 kDa) and β (190 kDa). Myosin-18Aα contains a lysine- and glutamic acid-rich (KE-rich) sequence at the extreme N terminus, followed by a PDZ domain (37,57). Myosin-18Aβ lacks the KE-rich sequence and the PDZ domain and, instead, has a short leading sequence upstream of the motor (37). Both isoforms have a predicted canonical IQ calmodulin-binding motif followed by a coiled-coil tail, analogous to the tail of myosin-2. A third isoform of Myo18A, p110 myosin, was identified in macrophages, which may come about through post-translational processing of Myo18Aα or β via the phosphorylation of tyrosine residues after the induction of macrophage differentiation by macrophage colony-stimulating factor-1 (CSF-1) treatment (61). At the cell level, myosin 18A participates in cytoskeleton organization (51), Golgi budding (52,53) and DNA-damaged-induced Golgi dispersion by its association with F-actin and Golgi Phosphoprotein 3 (GOLPH3) in epithelial cells (54) but its specific role in NK cells was not shown yet.

Myosin 18A was reported as a receptor for the surfactant protein A (SP-A) (40), a collectin present in human lung (62), blood (63), intestinal tract (64) and skin (65), that participates in the elimination of pathogens (43). SP-A has also been shown to strongly stimulate the anti-tumor immunity in a xenograft mouse model (42). Tumor cells transduced with SP-A grew slower than those transduced with vector alone. This anti-tumor effect of SP-A was entirely dependent on NK cells in vivo (42) although the exact mechanism remained unknown. Our data support the hypothesis that this major anti-tumor effect of SP-A in vivo is mediated by its interaction with Myo18A on NK cells.

The use of monoclonal antibodies modulating the NK cell antitumor function is a fastgrowing field of research. On the one side, monoclonal antibodies able to induce antibody-dependent cell cytotoxicity by targeting both the cancer cell and the FcγRIIIA/CD16 activating receptor present on NK cells have revolutionized the management of lymphoma (66-69) and human cancer (70,71). On the other, not all malignancies have an identified druggable target, and some cancers with an identified target escape therapeutic antibodies. In this setting, strategies aimed at increasing the efficacy of monoclonal antibodies are promising. Stimulation of the CD137 (4-1BB) receptor present on NK cells has been shown able to increase the efficacy of cetuximab (26), trastuzumab (27) and rituximab (28) in both in vitro and in vivo models of human cancer. The use of monoclonal antibodies that modulate the expression of CD137 at the NK cell surface, such as DY12 as shown in the present work, could be interesting in this setting. In conclusion, the NK cell activating receptor Myo18A appears as a very promising target in the field of the immunotherapy of human cancer and hematological malignancies.

### REFERENCES

Throughout this application, various references describe the state of the art to which this invention pertains. The disclosures of these references are hereby incorporated by reference into the present disclosure.
1. Rosenberg E B, Herberman R B, Levine P H, Halterman R H, McCoy J L, Wunderlich J R. Lymphocyte cytotoxicity reactions to leukemia-associated antigens in identical twins. Int J Cancer J Int Cancer. 15 mai 1972; 9(3):648-58.
2. Kiessling R, Klein E, Wigzell H. « Natural » killer cells in the mouse. I. Cytotoxic cells with specificity for mouse Moloney leukemia cells. Specificity and distribution according to genotype. Eur J Immunol. févr 1975; 5(2):112-7.
3. Kiessling R, Klein E, Pross H, Wigzell H. « Natural » killer cells in the mouse. II. Cytotoxic cells with specificity for mouse Moloney leukemia cells. Characteristics of the killer cell. Eur J Immunol. févr 1975; 5(2):117-21.
4. Takasugi M, Mickey M R, Terasaki P I. Reactivity of lymphocytes from normal persons on cultured tumor cells. Cancer Res. nov 1973; 33(11):2898-902.
5. Cerwenka A, Lanier L L. Natural killer cells, viruses and cancer. Nat Rev Immunol. October 2001; 1(1):41-9.
6. Caligiuri M A. Human natural killer cells. Blood. 1 wilt 2008; 112(3):461-9.
7. Moffett-King A. Natural killer cells and pregnancy. Nat Rev Immunol. September 2002; 2(9):656-63.
8. Hercend T, Griffin J D, Bensussan A, Schmidt R E, Edson M A, Brennan A, et al. Generation of monoclonal antibodies to a human natural killer clone. Characterization of two natural killer-associated antigens, NKH1A and NKH2, expressed on subsets of large granular lymphocytes. J Clin Invest. mars 1985; 75(3):932-43.
9. Lanier L L. Up on the tightrope: natural killer cell activation and inhibition. Nat Immunol. mai 2008; 9(5):495-502.
10. Melder R J, Koenig G C, Witwer B P, Safabakhsh N, Munn L L, Jain R K. During angiogenesis, vascular endothelial growth factor and basic fibroblast growth factor regulate natural killer cell adhesion to tumor endothelium. Nat Med. September 1996; 2(9):992-7.
11. Riteau B, Barber D F, Long E O. Vav1 phosphorylation is induced by beta2 integrin engagement on natural killer cells upstream of actin cytoskeleton and lipid raft reorganization. J Exp Med. 4 wilt 2003; 198(3):469-74.
12. Watzl C, Long E O. Natural killer cell inhibitory receptors block actin cytoskeleton-dependent recruitment of 2B4 (CD244) to lipid rafts. J Exp Med. 6 janv 2003; 197(1):77-85.
13. Davis D M, Chiu I, Fassett M, Cohen G B, Mandelboim O, Strominger J L. The human natural killer cell immune synapse. Proc Natl Acad Sci USA. 21 Dec. 1999; 96(26):15062-7.
14. Fütterer K, Wong J, Grucza R A, Chan A C, Waksman G. Structural basis for Syk tyrosine kinase ubiquity in signal transduction pathways revealed by the crystal structure of its regulatory SH2 domains bound to a dually phosphorylated ITAM peptide. J Mol Biol. 21 wilt 1998; 281(3):523-37.
15. Mandelboim O, Lieberman N, Lev M, Paul L, Arnon T I, Bushkin Y, et al. Recognition of haemagglutinins on virus-infected cells by NKp46 activates lysis by human NK cells. Nature. 22 févr 2001; 409(6823):1055-60.
16. Vitale M, Bottino C, Sivori S, Sanseverino L, Castriconi R, Marcenaro E, et al. NKp44, a novel triggering surface molecule specifically expressed by activated natural killer cells, is involved in non-major histocompatibility complex-restricted tumor cell lysis. J Exp Med. 15 juin 1998; 187(12):2065-72.
17. Pende D, Parolini S, Pessino A, Sivori S, Augugliaro R, Morelli L, et al. Identification and molecular characterization of NKp30, a novel triggering receptor involved in natural cytotoxicity mediated by human natural killer cells. J Exp Med. 15 Nov. 1999; 190(10):1505-16.
18. Bauer S, Groh V, Wu J, Steinle A, Phillips J H, Lanier L L, et al. Activation of NK cells and T cells by NKG2D, a receptor for stress-inducible MICA. Science. 30 juill 1999; 285(5428):727-9.
19. Moretta A, Tambussi G, Bottino C, Tripodi G, Merli A, Ciccone E, et al. A novel surface antigen expressed by a subset of human CD3-CD16+ natural killer cells. Role in cell activation and regulation of cytolytic function. J Exp Med. 1 mars 1990; 171(3):695-714.
20. Moretta A, Bottino C, Pende D, Tripodi G, Tambussi G, Viale O, et al. Identification of four subsets of human CD3-CD16+ natural killer (NK) cells by the expression of clonally distributed functional surface molecules: correlation between subset assignment of NK clones and ability to mediate specific alloantigen recognition. J Exp Med. 1 Dec. 1990; 172(6):1589-98.
21. Brown M H, Boles K, van der Merwe P A, Kumar V, Mathew P A, Barclay A N. 2B4, the natural killer and T cell immunoglobulin superfamily surface protein, is a ligand for CD48. J Exp Med. 7 Dec. 1998; 188(11):2083-90.
22. Le Bouteiller P, Barakonyi A, Giustiniani J, Lenfant F, Marie-Cardine A, Aguerre-Girr M, et al. Engagement of CD160 receptor by HLA-C is a triggering mechanism used by circulating natural killer (NK) cells to mediate cytotoxicity. Proc Natl Acad Sci USA. 24 déc 2002; 99(26):16963-8.
23. Bensussan A, Gluckman E, el Marsafy S, Schiavon V, Mansur I G, Dausset J, et al. BY55 monoclonal antibody delineates within human cord blood and bone marrow lymphocytes distinct cell subsets mediating cytotoxic activity. Proc Natl Acad Sci USA. 13 Sep. 1994; 91(19):9136-40.
24. Melero I, Johnston J V, Shufford W W, Mittler R S, Chen L. NK1.1 cells express 4-1BB (CDw137) costimulatory molecule and are required for tumor immunity elicited by anti-4-1BB monoclonal antibodies. Cell Immunol. 15 Dec. 1998; 190(2):167-72.
25. Lin W, Voskens C J, Zhang X, Schindler D G, Wood A, Burch E, et al. Fc-dependent expression of CD137 on human NK cells: insights into « agonistic » effects of anti-CD137 monoclonal antibodies. Blood. 1 wont 2008; 112(3):699-707.
26. Kohrt H E, Colevas A D, Houot R, Weiskopf K, Goldstein M J, Lund P, et al. Targeting CD137 enhances the efficacy of cetuximab. J Clin Invest. 2 juin 2014; 124(6):2668-82.
27. Kohrt H E, Houot R, Weiskopf K, Goldstein M J, Scheeren F, Czerwinski D, et al. Stimulation of natural killer cells with a CD137-specific antibody enhances trastuzumab efficacy in xenotransplant models of breast cancer. J Clin Invest. 1 mars 2012; 122(3):1066-75.
28. Kohrt H E, Houot R, Goldstein M J, Weiskopf K, Alizadeh A A, Brody J, et al. CD137 stimulation enhances the antilymphoma activity of anti-CD20 antibodies. Blood. 24 févr 2011; 117(8):2423-32.
29. Benson D M, Caligiuri M A. Killer immunoglobulin-like receptors and tumor immunity. Cancer Immunol Res. févr 2014; 2(2):99-104.
30. Braud V M, Allan D S, O'Callaghan C A, Soderstrom K, D'Andrea A, Ogg G S, et al. HLA-E binds to natural killer cell receptors CD94/NKG2A, B and C. Nature. 19 févr 1998; 391(6669):795-9.
31. Colonna M, Samaridis J. Cloning of immunoglobulin-superfamily members associated with HLA-C and HLA-B recognition by human natural killer cells. Science. 21 avr 1995; 268(5209):405-8.
32. Burshtyn D N, Scharenberg A M, Wagtmann N, Rajagopalan S, Berrada K, Yi T, et al. Recruitment of tyrosine phosphatase HCP by the killer cell inhibitor receptor. Immunity. janv 1996; 4(1):77-85.
33. Mason T, André P, Bensussan A. Leukocyte Typing VII. White Cell Differentiation Antigens. Oxford University Press. 2002.692-693 p.
34. Giustiniani J, Marie-Cardine A, Bensussan A. A Soluble Form of the MHC Class I-Specific CD160 Receptor Is Released from Human Activated NK Lymphocytes and Inhibits Cell-Mediated Cytotoxicity. J Immunol. 2 janv 2007; 178(3):1293-300.
35. Dufresne-Martin G, Lemay J-F, Lavigne P, Klarskov K. Peptide mass fingerprinting by matrix-assisted laser desorption ionization mass spectrometry of proteins detected by immunostaining on nitrocellulose. Proteomics. janv 2005; 5(1):55-66.
36. Mansur I-G, Schiavon V, Giustiniani J, Bagot M, Bensussan A, Marie-Cardine A. Engagement of IL-1 receptor accessory protein (IL-1RAcP) with the monoclonal antibody AY19 provides co-activating signals and prolongs the CD2-induced proliferation of peripheral blood lymphocytes. Immunol Lett. 30 Sep. 2011; 139(1-2):52-7.
37. Mori K, Furusawa T, Okubo T, Inoue T, Ikawa S, Yanai N, et al. Genome structure and differential expression of two isoforms of a novel PDZ-containing myosin (MysPDZ) (Myo18A). J Biochem (Tokyo). avr 2003; 133(4):405-13.
38. Yu J, Mao H C, Wei M, Hughes T, Zhang J, Park I, et al. CD94 surface density identifies a functional intermediary between the CD56bright and CD56dim human NK-cell subsets. Blood. 14 janv 2010; 115(2):274-81.
39. Saeki H, Moore A M, Brown M J, Hwang S T. Cutting Edge: Secondary Lymphoid-Tissue Chemokine (SLC) and CC Chemokine Receptor 7 (CCR7) Participate in the Emigration Pathway of Mature Dendritic Cells from the Skin to Regional Lymph Nodes. J Immunol. 3 janv 1999; 162(5):2472-5.
40. Yang C-H, Szeliga J, Jordan J, Faske S, Sever-Chroneos Z, Dorsett B, et al. Identification of the surfactant protein A receptor 210 as the unconventional myosin 18A. J Biol Chem. 14 Oct. 2005; 280(41):34447-57.
41. Samten B, Townsend J C, Sever-Chroneos Z, Pasquinelli V, Barnes P F, Chroneos Z C. An antibody against the surfactant protein A (SP-A)-binding domain of the SP-A receptor inhibits T cell-mediated immune responses to Mycobacterium tuberculosis. J Leukoc Biol. juill 2008; 84(1):115-23.
42. Mitsuhashi A, Goto H, Kuramoto T, Tabata S, Yukishige S, Abe S, et al. Surfactant protein A suppresses lung cancer progression by regulating the polarization of tumor-associated macrophages. Am J Pathol. mai 2013; 182(5):1843-53.
43. McNeely T B, Coonrod J D. Aggregation and opsonization of type A but not type B Hemophilus influenzae by surfactant protein A. Am J Respir Cell Mol Biol. juill 1994; 11(1):114-22.
44. Zhao S, Zhang H, Xing Y, Natkunam Y. CD137 ligand is expressed in primary and secondary lymphoid follicles and in B-cell lymphomas: diagnostic and therapeutic implications. Am J Surg Pathol. févr 2013; 37(2):250-8.
45. Rak G D, Mace E M, Banerjee P P, Svitkina T, Orange J S. Natural killer cell lytic granule secretion occurs through a pervasive actin network at the immune synapse. PLoS Biol. September 2011; 9(9):e1001151.
46. Brown ACN, Oddos S, Dobbie I M, Alakoskela J-M, Parton R M, Eissmann P, et al. Remodelling of cortical actin where lytic granules dock at natural killer cell immune synapses revealed by super-resolution microscopy. PLoS Biol. September 2011; 9(9):e1001152.
47. Hsu R-M, Tsai M-H, Hsieh Y-J, Lyu P-C, Yu J-S. Identification of MYO18A as a novel interacting partner of the PAK2/betaPIX/GIT1 complex and its potential function in modulating epithelial cell migration. Mol Biol Cell. 15 janv 2010; 21(2):287-301.
48. Stebbins C C, Watzl C, Billadeau D D, Leibson P J, Burshtyn D N, Long E O. Vav1 dephosphorylation by the tyrosine phosphatase SHP-1 as a mechanism for inhibition of cellular cytotoxicity. Mol Cell Biol. September 2003; 23(17):6291-9.
49. Mahmood S, Kanwar N, Tran J, Zhang M-L, Kung SKP. SHP-1 phosphatase is a critical regulator in preventing natural killer cell self-killing. PloS One. 2012; 7(8):e44244.
50. Purdy A K, Campbell K S. SHP-2 expression negatively regulates NK cell function. J Immunol Baltim Md. 1950.1 déc 2009; 183(11):7234-43.
51. Taft M H, Behrmann E, Munske-Weidemann L-C, Thiel C, Raunser S, Manstein D J. Functional characterization of human myosin-18A and its interaction with F-actin and GOLPH3. J Biol Chem. 18 Oct. 2013; 288(42):30029-41.
52. Dippold H C, Ng M M, Farber-Katz S E, Lee S-K, Kerr M L, Peterman M C, et al. GOLPH3 bridges phosphatidylinositol-4-phosphate and actomyosin to stretch and shape the Golgi to promote budding. Cell. 16 Oct. 2009; 139(2):337-51.
53. Ng M M, Dippold H C, Buschman M D, Noakes C J, Field S J. GOLPH3L antagonizes GOLPH3 to determine Golgi morphology. Mol Biol Cell. mars 2013; 24(6):796-808.
54. Farber-Katz S E, Dippold H C, Buschman M D, Peterman M C, Xing M, Noakes C J, et al. DNA damage triggers Golgi dispersal via DNA-P K and GOLPH3. Cell. 30 janv 2014; 156(3):413-27.
55. Hartman M A, Spudich J A. The myosin superfamily at a glance. J Cell Sci. 1 avr 2012; 125(Pt 7):1627-32.
56. Andzelm M M, Chen X, Krzewski K, Orange J S, Strominger J L. Myosin H A is required for cytolytic granule exocytosis in human NK cells. J Exp Med. 1 Oct. 2007; 204(10):2285-91.
57. Furusawa T, Ikawa S, Yanai N, Obinata M. Isolation of a novel PDZ-containing myosin from hematopoietic supportive bone marrow stromal cell lines. Biochem Biophys Res Commun. 2 avr 2000; 270(1):67-75.
58. Nakano T, Tani M, Nishioka M, Kohno T, Otsuka A, Ohwada S, et al. Genetic and epigenetic alterations of the candidate tumor-suppressor gene MYO18B, on chromosome arm 22q, in colorectal cancer. Genes Chromosomes Cancer. juin 2005; 43(2):162-71.
59. Nishioka M, Kohno T, Tani M, Yanaihara N, Tomizawa Y, Otsuka A, et al. MYO18B, a candidate tumor suppressor gene at chromosome 22q12.1, deleted, mutated, and methylated in human lung cancer. Proc Natl Acad Sci USA. 17 Sep. 2002; 99(19):12269-74.
60. Yanaihara N, Nishioka M, Kohno T, Otsuka A, Okamoto A, Ochiai K, et al. Reduced expression of MYO18B, a candidate tumor-suppressor gene on chromosome arm 22q, in ovarian cancer. Int J Cancer J Int Cancer. 20 Oct. 2004; 112(1):150-4.
61. Cross M, Csar X F, Wilson N J, Manes G, Addona T A, Marks D C, et al. A novel 110 kDa form of myosin XVIIIA (MysPDZ) is tyrosine-phosphorylated after colony-stimulating factor-1 receptor signalling. Biochem J. 15 mai 2004; 380(Pt 1):243-53.
62. Haagsman H P, Hawgood S, Sargeant T, Buckley D, White R T, Drickamer K, et al. The major lung surfactant protein, SP 28-36, is a calcium-dependent, carbohydrate-binding protein. J Biol Chem. 15 Oct. 1987; 262(29):13877-80.
63. Kuroki Y, Tsutahara S, Shijubo N, Takahashi H, Shiratori M, Hattori A, et al. Elevated levels of lung surfactant protein A in sera from patients with idiopathic pulmonary fibrosis and pulmonary alveolar proteinosis. Am Rev Respir Dis. mars 1993; 147(3):723-9.
64. Rubio S, Lacaze-Masmonteil T, Chailley-Heu B, Kahn A, Bourbon J R, Ducroc R. Pulmonary surfactant protein A (SP-A) is expressed by epithelial cells of small and large intestine. J Biol Chem. 19 mai 1995; 270(20):12162-9.
65. Aiad HAS, El-Farargy S M, Soliman M M, El-Wahed Gaber M A, El-Aziz Othman S A. Immunohistochemical staining of surfactant proteins A and B in skin of psoriatic patients before and after narrow-band UVB phototherapy. Am J Clin Dermatol. 1 Oct. 2012; 13(5):341-8.
66. Coiffier B, Lepage E, Briére J, Herbrecht R, Tilly H, Bouabdallah R, et al. CHOP Chemotherapy plus Rituximab Compared with CHOP Alone in Elderly Patients with Diffuse Large-B-Cell Lymphoma. N Engl J Med. 24 janv 2002; 346(4):235-42.
67. Bouaziz J-D, Ortonne N, Giustiniani J, Schiavon V, Huet D, Bagot M, et al. Circulating natural killer lymphocytes are potential cytotoxic effectors against autologous malignant cells in sezary syndrome patients. J Invest Dermatol. dec 2005; 125(6):1273-8.
68. De Masson A, Guitera P, Brice P, Moulonguet I, Mouly F, Bouaziz J-D, et al. Long-term efficacy and safety of alemtuzumab in advanced primary cutaneous T-cell lymphomas. Br J Dermatol. mars 2014; 170(3):720-4.
69. Marie-Cardine A, Viaud N, Thonnart N, Joly R, Chanteux S, Gauthier L, et al. IPH4102, a humanized KIR3DL2 antibody with potent activity against cutaneous T-cell lymphoma. Cancer Res. 1 Nov. 2014; 74(21):6060-70.
70. Cunningham D, Humblet Y, Siena S, Khayat D, Bleiberg H, Santoro A, et al. Cetuximab Monotherapy and Cetuximab plus Irinotecan in Irinotecan-Refractory Metastatic Colorectal Cancer. N Engl J Med. 22 juill 2004; 351(4):337-45.
71. Romond E H, Perez E A, Bryant J, Suman V J, Geyer C E, Davidson N E, et al. Trastuzumab plus Adjuvant Chemotherapy for Operable HER2-Positive Breast Cancer. N Engl J Med. 20 Oct. 2005; 353(16):1673-84.

1. A method of enhancing NK cell killing activities in a subject in need thereof comprising administering to the subject a therapeutically effective amount of a compound capable of stimulating CD245 on NK cells, wherein said compound is a chimeric, humanized or human antibody having specificity to CD245.

2. (canceled)

3. The method of claim 1 wherein the antibody is selected from the group consisting of Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like.

4. The method of claim 1 wherein the antibody is a monoclonal antibody.

5. (canceled)

6. The method of claim 1 wherein the antibody is a single domain antibody.

7. The method of claim 1 wherein the antibody comprises human heavy chain constant regions sequences but will not deplete NK cells to which they are bound.

8. The method of claim 1 wherein the antibody does not comprise an Fc domain capable of substantially binding to a FcgammaRIIIA (CD16) polypeptide.

9. The method of claim 1 wherein the antibody lacks an Fc domain or comprises an Fc domain of IgG2 or IgG4 isotype.

10. The method of claim 1 wherein the antibody is a multispecific antibody comprising a first antigen binding site having specificity for CD245 and at least one second antigen binding site.

11. The method of claim 1 wherein the antibody is a multispecific antibody comprising a first antigen binding site having specificity for CD245 and at least one second antigen binding site, wherein the second antigen-binding site binds to an antigen that is expressed by a cancer cell or a cell infected by a virus or a bacterium.

12. A method of treating a cancer or an infectious disease in a subject in need thereof comprising administering to the subject a therapeutically effective amount of a compound capable of stimulating CD245 on NK cells, wherein said compound is a chimeric, humanized or human antibody having specificity to CD245.

13. The method of claim 12 wherein the compound capable of stimulating CD245 on NK cells is used in combination with an antibody having specificity for CD137.

14. The method of claim 12 wherein the compound capable of stimulating CD245 on NK cells is used in combination with a chemotherapeutic agent, a targeted cancer therapy, an immunotherapeutic agent, or an antibody that is specific for a costimulatory molecule.

15. The method of claim 12 wherein the compound capable of stimulating CD245 on NK cells is used in combination with a second agent that induces, via ADCC, the death of a cell expressing an antigen to which the second agent binds.

16. A method of enhancing NK cell antibody-dependent cellular cytotoxicity (ADCC) of an antibody in a subject in need thereof comprising administering to the subject the antibody, and administering to the subject a compound capable of stimulating CD245 on NK cells, wherein said compound is a chimeric, humanized or human antibody having specificity to CD245.

17. The method according to claim 16, wherein said method is a method of treating cancer in a subject in need thereof and comprises administering to the subject a first antibody selective for a cancer cell antigen, and administering to the subject a compound capable of stimulating CD245 on NK cells.

18. The method of claim 1 wherein the antibody does not comprise an Fc portion that induces antibody dependent cellular cytotoxicity (ADCC).

19. The method of claim 12 wherein the antibody is a multispecific antibody comprising a first antigen binding site having specificity for CD245 and at least one second antigen binding site.

20. The method of claim 12 wherein the antibody is a multispecific antibody comprising a first antigen binding site having specificity for CD245 and at least one second antigen binding site, wherein the second antigen-binding site binds to an antigen that is expressed by a cancer cell or a cell infected by a virus or a bacterium.


SEQUENCE LISTING (1 sequence)

SEQ ID NO: 1 - protein, 2054 aa, Homo sapiens
MFNLMKKDKDKDGGRKEKKEKKEKKERMSAAELRSLEEMSLRRGFFNLNRSSKRESKTRLEISNPIPIKVASGSDLHLTDIDSDSNRGSVILDSGHLSTASSSDDLKGEEGSFRGSVLQRAAKFGSLAKQNSQMIVKRFSFSQRSRDESASETSTPSEHSAAPSPQVEVRTLEGQLVQHPGPGIPRPGHRSRAPELVTKKFPVDLRLPPVVPLPPPTLRELELQRRPTGDFGFSLRRTTMLDRGPEGQACRRVVHFAEPGAGTKDLALGLVPGDRLVEINGHNVESKSRDEIVEMIRQSGDSVRLKVQPIPELSELSRSWLRSGEGPRREPSDAKTEEQIAAEEAWNETEKVWLVHRDGFSLASQLKSEELNLPEGKVRVKLDHDGAILDVDEDDVEKANAPSCDRLEDLASLVYLNESSVLHTLRQRYGASLLHTYAGPSLLVLGPRGAPAVYSEKVMHMFKGCRREDMAPHIYAVAQTAYRAMLMSRQDQSIILLGSSGSGKTTSCQHLVQYLATIAGISGNKVFSVEKWQALYTLLEAFGNSPTIINGNATRFSQILSLDFDQAGQVASASIQTMLLEKLRVARRPASEATFNVFYYLLACGDGTLRTELHLNHLAENNVFGIVPLAKPEEKQKAAQQFSKLQAAMKVLGISPDEQKACWFILAAIYHLGAAGATKEAAEAGRKQFARHEWAQKAAYLLGCSLEELSSAIFKHQHKGGTLQRSTSFRQGPEESGLGDGTGPKLSALECLEGMAAGLYSELFTLLVSLVNRALKSSQHSLCSMMIVDTPGFQNPEQGGSARGASFEELCHNYTQDRLQRLFHERTFVQELERYKEENIELAFDDLEPPTDDSVAAVDQASHQSLVRSLARTDEARGLLWLLEEEALVPGASEDTLLERLFSYYGPQEGDKKGQSPLLHSSKPHHFLLGHSHGTNWVEYNVTGWLNYTKQNPATQNAPRLLQDSQKKIISNLFLGRAGSATVLSGSIAGLEGGSQLALRRATSMRKTFTTGMAAVKKKSLCIQMKLQVDALIDTIKKSKLHFVHCFLPVAEGWAGEPRSASSRRVSSSSELDLPSGDHCEAGLLQLDVPLLRTQLRGSRLLDAMRMYRQGYPDHMVFSEFRRRFDVLAPHLTKKHGRNYIVVDERRAVEELLECLDLEKSSCCMGLSRVFFRAGTLARLEEQRDEQTSRNLTLFQAACRGYLARQHFKKRKIQDLAIRCVQKNIKKNKGVKDWPWWKLFTTVRPLIEVQLSEEQIRNKDEEIQQLRSKLEKAEKERNELRLNSDRLESRISELTSELTDERNTGESASQLLDAETAERLRAEKEMKELQTQYDALKKQMEVMEMEVMEARLIRAAEINGEVDDDDAGGEWRLKYERAVREVDFTKKRLQQEFEDKLEVEQQNKRQLERRLGDLQADSEESQRALQQLKKKCQRLTAELQDTKLHLEGQQVRNHELEKKQRRFDSELSQAHEEAQREKLQREKLQREKDMLLAEAFSLKQQLEEKDMDIAGFTQKVVSLEAELQDISSQESKDEASLAKVKKQLRDLEAKVKDQEEELDEQAGTIQMLEQAKLRLEMEMERMRQTHSKEMESRDEEVEEARQSCQKKLKQMEVQLEEEYEDKQKVLREKRELEGKLATLSDQVNRRDFESEKRLRKDLKRTKALLADAQLMLDHLKNSAPSKREIAQLKNQLEESEFTCAAAVKARKAMEVEIEDLHLQIDDIAKAKTALEEQLSRLQREKNEIQNRLEEDQEDMNELMKKHKAAVAQASRDLAQINDLQAQLEEANKEKQELQEKLQALQSQVEFLEQSMVDKSLVSRQEAKIRELETRLEFERTQVKRLESLASRLKENMEKLTEERDQRIAAENREKEQNKRLQRQLRDTKEEMGELARKEAEASRKKHELEMDLESLEAANQSLQADLKLAFKRIGDLQAAIEDEMESDENEDLINSLQDMVTKYQKRKNKLEGDSDVDSELEDRVDGVKSWLSKNKGPSKAASDDGSLKSSSPTSYWKSLAPDRSDDEHDPLDNTSRPRYSHSYLSDSDTEAKLTETNA